(19) United States
(12) Patent Application Publication
(10) Pub. No.: US 2024/0295560 A1
(43) Pub. Date: Sep. 5, 2024

(54) PANCREATIC CANCER DIAGNOSIS ASSISTANCE METHOD AND PHARMACEUTICAL COMPOUND FOR TREATING PANCREATIC CANCER

(71) Applicant: TOKYO METROPOLITAN GERIATRIC HOSPITAL AND INSTITUTE OF GERONTOLOGY, Itabashi-ku, Tokyo (JP)

(72) Inventors: Toshiyuki ISHIWATA, Itabashi-ku, Tokyo (JP); Norihiko SASAKI, Itabashi-ku, Tokyo (JP)

(73) Assignee: TOKYO METROPOLITAN GERIATRIC HOSPITAL AND INSTITUTE OF GERONTOLOGY, Itabashi-ku, Tokyo (JP)

(21) Appl. No.: 17/802,767

(22) PCT Filed: Feb. 26, 2021
(86) PCT No.: PCT/JP2021/007390

§ 371 Date: Dec. 22, 2022

(30) Foreign Application Priority Data
Feb. 28, 2020 (JP) 2020-032798

(51) Int. Cl. G01N 33/574; A61K 41/00; A61K 47/68; A61P 35/00

(52) U.S. Cl. CPC G01N 33/57438; A61K 41/0071; A61K 47/6859; A61P 35/00; G01N 2333/705

(57) ABSTRACT

The object of the present invention is to provide a method for early diagnosing pancreatic cancer and a pharmaceutical composition for treating pancreatic cancer. The object can be solved by a method for assisting a diagnosis of pancreatic cancer, comprising the steps of: (1) bringing a sample derived from a subject suspected of having pancreatic cancer into contact with an antibody specific to ganglioside GM2 and/or an antibody specific to ganglioside GD1a, to obtain an antigen-antibody complex, and (2) detecting the antigen-antibody complex; and a pharmaceutical composition for treating pancreatic cancer, comprising a GM2 immune complex wherein an anticancer drug or a photosensitizer is bound to an antibody specific to ganglioside GM2, and/or a GD1a immune complex wherein an anticancer drug or a photosensitizer is bound to an antibody specific to ganglioside GD1a.

### TECHNICAL FIELD

The present invention relates to a method for assisting a diagnosis of pancreatic cancer and a pharmaceutical composition for treating pancreatic cancer. According to the present invention, the pancreatic cancer can be diagnosed and can be treated.

### BACKGROUND ART

Pancreatic cancer is increasing rapidly, especially in the elderly. In many cases, the cancer has already invaded and metastasized at the time of discovery, and only about 20% of people can undergo surgery (Non-Patent Literature 1 or 2). In addition, the survival rate is less than 10% (Non-Patent Literature 3 or 4), and thus the development of early diagnosis and new treatments for pancreatic cancer are required.

### CITATION LIST

### Non-Patent Literature

[NON-PATENT LITERATURE 1] Future Oncology (the United Kingdom) 2016, vol 12, p 669-685
[NON-PATENT LITERATURE 2] Future Oncology (the United Kingdom) 2016, vol 12, p 1929-1946
[NON-PATENT LITERATURE 3] A Cancer Journal for Clinicians (U.S.A.) 2019, vol 69, p 7-34
[NON-PATENT LITERATURE 4] World Journal of Gastroenterology (U.S.A.) 2018, vol 24, p 4846-4861

### SUMMARY OF INVENTION

### Technical Problem

The object of the present invention is to provide a method for early diagnosing pancreatic cancer and a pharmaceutical composition for treating pancreatic cancer.

### Solution to Problem

The present inventors have conducted intensive studies on the method for early diagnosing pancreatic cancer and the pharmaceutical composition for treating pancreatic cancer, as a result, surprisingly found that ganglioside GM2 or GD1a is expressed in the pancreatic cancer. Further, the inventors found that the expression of GM2 or GD1a in cells and cancer tissues correlates with the malignancy of cancer such as rapid growth rate and stage thereof. Furthermore, by targeting ganglioside GM2 or GD1a, the inventors found that pancreatic cancer can be treated by binding an anticancer drug or a photosensitizer to an antibody.

The present invention is based on the above findings.

Accordingly, the present invention relates to:
[1] a method for assisting a diagnosis of pancreatic cancer, comprising the steps of: (1) bringing a sample derived from a subject suspected of having pancreatic cancer into contact with an antibody specific to ganglioside GM2 and/or an antibody specific to ganglioside GD1a, to obtain an antigen-antibody complex, and (2) detecting the antigen-antibody complex,
[2] a method for assisting prediction of malignancy of pancreatic cancer, comprising the steps of: (1) bringing a sample derived from a pancreatic cancer patient into contact with an antibody specific to ganglioside GM2 and/or an antibody specific to ganglioside GD1a, to obtain an antigen-antibody complex, and (2) detecting the antigen-antibody complex,
[3] a kit for diagnosing pancreatic cancer, or predicting malignancy of pancreatic cancer, comprising an antibody specific to ganglioside GM2 and/or an antibody specific to ganglioside GD1a, and an agent for detecting an antigen-antibody complex(s) of the antibody and ganglioside GM2 and/or the antibody and ganglioside GD1a,
[4] a pharmaceutical composition for treating pancreatic cancer, comprising a GM2 immune complex wherein an anticancer drug or a photosensitizer is bound to an antibody specific to ganglioside GM2, and/or a GD1a immune complex wherein an anticancer drug or a photosensitizer is bound to an antibody specific to ganglioside GD1a,
[5] the pharmaceutical composition for treating pancreatic cancer of the item [4], wherein the photosensitizer is phthalocyanine,
[6] the pharmaceutical composition for treating pancreatic cancer of the item [4] or [5], wherein the antibody is a humanized antibody, and
[7] a method for determining a therapeutic effect of the pharmaceutical composition for treating pancreatic cancer of any one of the items [4] to [6].

The present specification discloses:
[8] a method for assisting a diagnosis of pancreatic cancer, comprising the steps of: (1) bringing a sample derived from a subject suspected of having pancreatic cancer into contact with an antibody specific to ganglioside GD1a, to obtain an antigen-antibody complex, and (2) detecting the antigen-antibody complex
[9] a method for assisting prediction of malignancy of pancreatic cancer, comprising the steps of: (1) bringing a sample derived from a pancreatic cancer patient into contact with an antibody specific to ganglioside GD1a, to obtain an antigen-antibody complex, and (2) detecting the antigen-antibody complex,
[10] a kit for diagnosing pancreatic cancer, or predicting malignancy of pancreatic cancer, comprising an antibody specific to ganglioside GD1a, and an agent for detecting an antigen-antibody complex(s) of the antibody and ganglioside GD1a
[11] a pharmaceutical composition for treating pancreatic cancer, comprising a GD1a immune complex wherein an anticancer drug or a photosensitizer is bound to an antibody specific to ganglioside GD1a,
[12] the pharmaceutical composition for treating pancreatic cancer of the item [11], wherein the photosensitizer is phthalocyanine
[13] the pharmaceutical composition for treating pancreatic cancer of the item or [12], wherein the antibody is a humanized antibody, and
[14] a method for determining a therapeutic effect of the pharmaceutical composition for treating pancreatic cancer of any one of the items to [13].

### Advantageous Effects of Invention

According to the method for assisting a diagnosis of pancreatic cancer of the present invention, the pancreatic cancer can be early diagnosed. According to the method for assisting prediction of malignancy of pancreatic cancer of the present invention, the malignancy of pancreatic cancer can be predicted. In addition, according to the pharmaceutical composition for treating pancreatic cancer of the present invention, the pancreatic cancer can be effectively treated.

### BRIEF DESCRIPTION OF THE DRAWINGS

FIG. 1 is figures showing the sugar chain structure of ganglioside GM2 (A) and ganglioside GD1a (B) schematically.

FIG. 2 is figures showing the method for assisting a diagnosis of pancreatic cancer wherein ganglioside positive cells are detected, and the treatment of pancreatic cancer by photoimmunotherapy, schematically.

FIG. 3 is figures showing the analysis of the GM2 positive cells in MIA PaCa-2 cells by flow cytometry (A), and the sorting of the GM2 positive cells in MIA PaCa-2 cells by cell sorter (B).

FIG. 4 is a graph showing the levels of GD1a in PANC-1 cells, T3M-4 cells, PK-59 cells, PK-45P cells, MIA PaCa-2 cells, PK-8 cells, PK-1 cells, and KP-4 cells derived from pancreatic cancer.

FIG. 5 is a graph showing the cell growth rate of GM2 positive cells and GM2 negative cells of MIA PaCa-2 cells.

FIG. 6 is graphs showing anticancer effects of gemcitabine, fluorouracil [5-FU], and abraxane, which are anticancer drug, against the GM2 positive cells or GM2 negative cells.

FIG. 7 is figures showing the influence of the expression of GM2 in MIA PaCa-2 cells by three-dimensional culture.

FIG. 8 is figures showing the influence of the expression of GM2 in MIA PaCa-2 cells by N-(5′-adamantane-1′-yl-methoxy)-pentyl-1-deoxynojirimycin (AMP-dNM) or MAP kinase inhibitor.

FIG. 9 is a graph showing the growth of GM2 expression cells in nude mice.

FIG. 10 is immunohistochemical staining photographs showing the GM2 expression in human pancreatic cancer tissues.

FIG. 11 is immunohistochemical staining photographs showing the GD1a expression in human pancreatic cancer tissues.

FIG. 12 is graphs showing the induction of cell death by targeting cells expressing GM2 (A) or GD1a (B) and by using an anti-GM2 or anti-GD1a antibody and a secondary antibody labeled with saporin.

### DESCRIPTION OF EMBODIMENTS

### [1] Method for Assisting Diagnosis of Pancreatic Cancer

The method for assisting a diagnosis of pancreatic cancer of the present invention comprises the steps of (1) bringing a sample derived from a subject suspected of having pancreatic cancer into contact with an antibody specific to ganglioside GM2 and/or an antibody specific to ganglioside GD1a, to obtain an antigen-antibody complex, and (2) detecting the antigen-antibody complex.

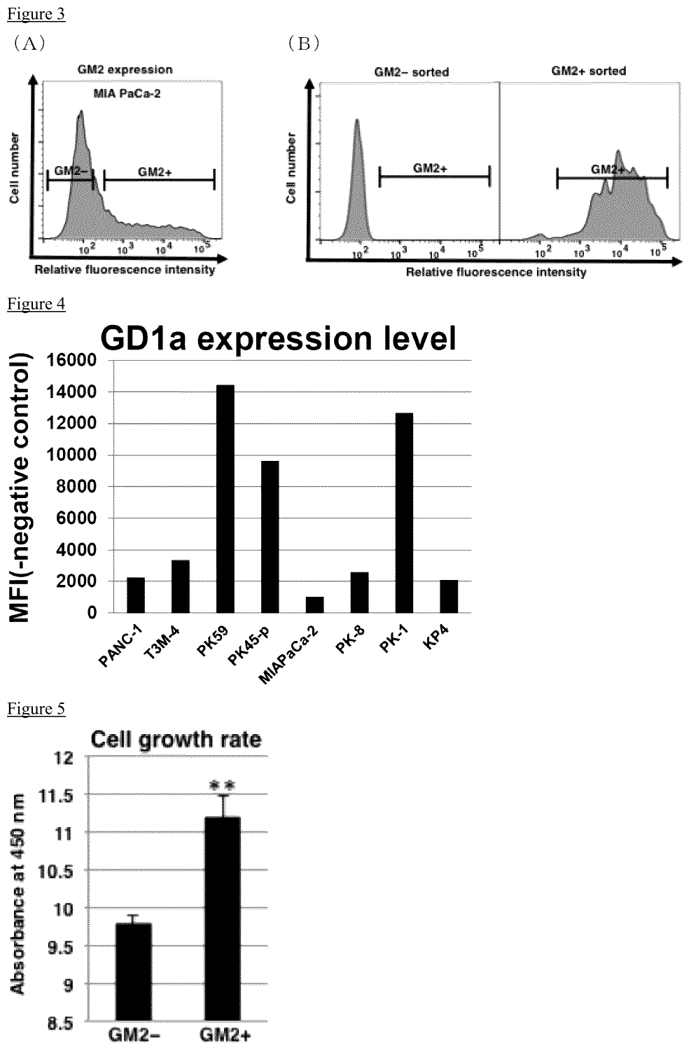

As shown in FIG. 2, by detecting GM2 or GD1a in a pancreatic tissue, serum, or the like of a patient suspected of having pancreatic cancer, early diagnosis of pancreatic cancer can be assisted.

### (Pancreatic Cancer)

90% or more of pancreatic cancers are pancreatic duct cancers formed in the cells of the pancreatic duct, and the pancreatic cancer usually refers to this pancreatic duct cancer. pancreatic cancer. Pancreatic cancer includes neuroendocrine tumors, intraductal papillary mucinous tumors, or the like. As risk factors for pancreatic cancer, there may be mentioned chronic pancreatitis, diabetes, obesity, and smoking.

### (Subject Suspected of Having Pancreatic Cancer)

In the method for assisting a diagnosis of pancreatic cancer of the present invention, a sample derived from a subject suspected of having pancreatic cancer is examined. The subject suspected of having pancreatic cancer is, for example, a patient with suspected symptoms of pancreatic cancer. As the symptoms of pancreatic cancer, there may be mentioned abdominal pain, loss of appetite, abdominal bloating, jaundice, lower back pain, back pain, or the like. It may also develop diabetes. The pancreatic cancer can be distinguished from other cancers by these symptoms.

The Subjects suspected of having pancreatic cancer include patients diagnosed with pancreatic cancer by a doctor or the like. This is because the method for assisting a diagnosis of pancreatic cancer of the present invention can be used as a diagnostic aid of a patient diagnosed with pancreatic cancer.

### (Samples)

The samples used in the present invention are not limited, as long as the samples are derived from the subjects suspected of having pancreatic cancer. For example, there may be mentioned pancreatic cells (including cultured cells derived from pancreas), pancreatic tissue, urine, blood, serum, plasma, lymph fluid, tissue fluid, spinal fluid, saliva, or sweat. That is, it is not particularly limited as long as it is a sample that may contain ganglioside GM2 or ganglioside GD1a.

### (Ganglioside GM2)

Ganglioside is a glycosphingolipid in which one or more sialic acids (N-acetylneuraminic acid: Neu5Ac, NANA) are bound on a sugar chain. 40 kinds or more of gangliosides have been found. GM2 is a ganglioside represented by GalNAcβ1→4(NeuAcα2→3)Galβ1→4Glcβ1→1Cer.

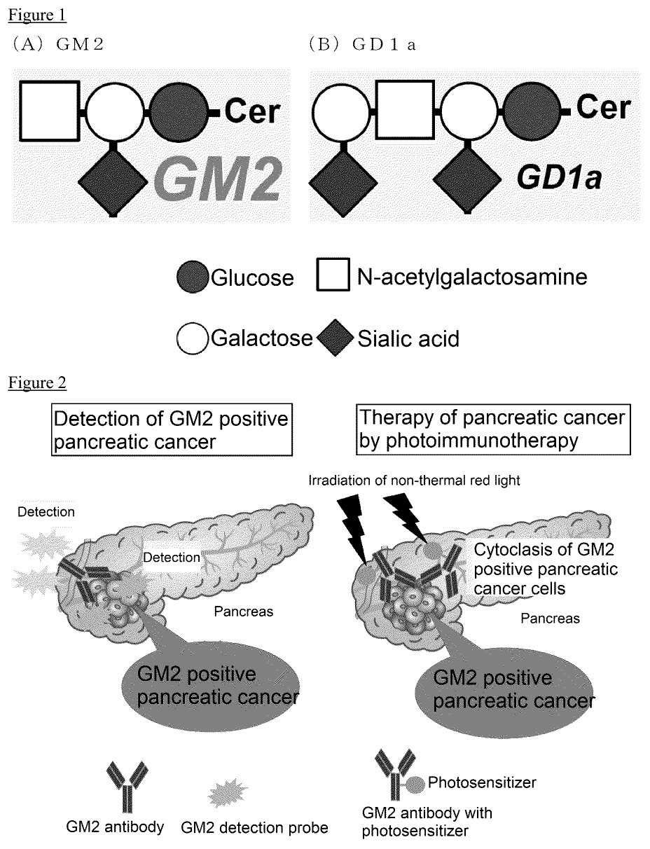

Specifically, as shown in FIG. 1(A), glucose, galactose, and N-acetylgalactosamine are bound in this order from the cell membrane, and sialic acid (N-acetylneuraminic acid) is bound as a side chain of galactose.

### (Ganglioside GD1a)

GD1a is also a type of ganglioside and is represented by NeuAcα2→3Galβ1→3GalNAcβ1→4(NeuAcα2→3)Galβ1→4Glcβ1→1Cer. Specifically, as shown in FIG. 1(B), glucose, galactose, N-acetylgalactosamine, and galactose are bound in this order from the cell membrane, and two sialic acids (N-acetylneuraminic acid) are bound as side chains of galactose.

### (Antibody)

An antibody used in the method for assisting a diagnosis of pancreatic cancer of the present invention is not particularly limited, as long as it specifically binds to ganglioside GM2 or ganglioside GD1a, but there may be mentioned monoclonal antibodies or polyclonal antibodies, or antigen-binding fragments thereof. The monoclonal antibody or polyclonal antibody may be produced according to known methods, except that ganglioside GM2 or ganglioside GD1a is used as an antigen for immunization. For example, the monoclonal antibody is prepared according to a cell fusion method of Kohler and Milstein (Nature, 256, 495-497, 1975). In a preparation of polyclonal antibody, for example, ganglioside GM2 or ganglioside GD1a is conjugated to BSA or KLH. The conjugated β3Gal-T4 or β3Gal-T5, or unconjugated β3Gal-T4 or β3Gal-T5 is emulsified in an appropriate adjuvant (for example, Freund's complete adjuvant), and rabbits are routinely immunized intradermally by using them. If an antibody titer in blood is increased, blood is collected and can be used as an antiserum or an antibody purified by the known methods.

Commercially available antibodies can be used as the antibodies described above. For example, the commercially available antibodies includes Anti-GM2 Monoclonal Antibody (TCI: A2576) or Anti-GD1a Monoclonal Antibody (TCI: A2507).

As the antigen-binding fragment of the antibody, there may be mentioned, for example, F(ab′)₂, Fab′, Fab, Fv, scFV, dsFV, nanobody, or the like. The antigen-binding fragment may be obtained by conventional methods, for example, by digesting the antibody using a protease (such as pepsin, papain, or the like) and purifying the resulting fragments by standard polypeptide isolation and purification methods. In addition, it can also be obtained by the genetically recombination method wherein antibody gene is incorporated into a vector and E. coli or the like is used.

In this specification, “antibody specific for ganglioside GM2” and “antibody specific for ganglioside GD1a” may sometimes mean antigen-binding fragments having antigen-binding sites of antibodies that specifically bind to GM2 or GD1a.

For example, when the method for assisting a diagnosis of pancreatic cancer of the present invention is an immunoassay using a labeled antibody, such as enzyme immunoassay, chemiluminescent immunoassay, fluorescence antibody technique, radioimmunoassay, or immunohistological staining method, the labeled antibody or antibody fragment conjugated by a labeling material can be contained therein. Specifically, the labeling material can include peroxidase, alkaline phosphatase, β-D-galactosidase, or glucose oxidase as an enzyme. In addition, in the case of an enzyme or a chemiluminescent substance, since they cannot develop a measurable signal by themselves, a substance corresponding to each enzyme or chemiluminescent substance is preferably used.

### «Contact Step 1»

In the contact step (1), a sample derived from a subject suspected of having pancreatic cancer is brought into contact with an antibody specific to ganglioside GM2 and/or an antibody specific to ganglioside GD1a, to obtain an antigen-antibody complex. In the antigen-antibody complex, GM2 or GD1a and the antibody are bound by an epitope and an antigen binding site of the antibody. As the method for assisting a diagnosis of pancreatic cancer of the present invention, there may be mentioned an enzyme immunoassay, a latex agglutination immunoassay, a chemiluminescent immunoassay, a fluorescent antibody method, a radioimmunoassay, an immunoprecipitation method, an immunohistological staining method, or a western blot. In either method, the antigen-antibody complex is formed.

### «Detection Step 2»

In the Detection step (2), the antigen-antibody complex is detected. A detecting means is not limited. For example, in the immunohistological staining method or the like, antibodies bound to GM2 antigen or GD1a antigen in pancreatic cells or pancreatic tissues may be captured. Specifically, the signal can be detected by labeling the antibody with an enzyme such as peroxidase and reacting it with chromogenic or luminescent substrates. In the fluorescent antibody method, the radioimmunoassay, and the like, antibodies are labeled with fluorescent substance such as fluorescein or radioactive substance, and fluorescence or radioactivity can be detected.

In the Western blotting, after electrophoresis, the antibody labeled with the enzyme of the antigen-antibody complex bound to nitrocellulose membranes can be detected by using chromogenic or luminescent substrates, to detect signals.

In either method, the antigen-antibody complex can be detected by using a secondary antibody against the antibody therein. In this case, the signal can be detected by labeling the secondary antibody with an enzyme, a fluorescent substance, or a radioactive substance.

When the method for assisting a diagnosis of pancreatic cancer of the present invention is carried out by an enzyme-linked immunosorbent assay using a solid phase, which is one of the enzyme immunoassay, there may be mentioned a sandwich method, and a competitive method, which are not limited, but the competitive method is preferred. This is because, in the sandwich method, the antigen is detected by sandwiching it between two antibodies. In GM2 and GD1a, it is sometimes difficult for two antibodies to bind to the antigen at the same time because the antigen is a small molecule.

In the competitive assay, the antibody that bind specifically to ganglioside GM2 or ganglioside GD1a is immobilized on ELISA plates, beads, magnetic particles, or the like. Next, samples containing GM2 or GD1a are mixed with GM2 or GD1a labeled with enzyme, or the like (labeled competitive antigen) for competition, and the whole is added to the ELISA plate. GM2 or GD1a in the sample can be measured by detecting the signal from the enzyme after the reaction. The signal becomes smaller when the amount of GM2 or GD1a in the sample is high and the signal becomes larger when the amount of GM2 or GD1a is low, and therefore the concentration of GM2 or GD1a in the sample can be determined from a standard curve.

The enzyme-linked immunosorbent assay is preferably used for liquid samples such as urine, blood, serum, plasma, lymph fluid, tissue fluid, marrow fluid, saliva, or sweat.

In the method for assisting a diagnosis of pancreatic cancer of the present invention, the pancreatic cancer can be detected by measuring the amount or concentration of ganglioside GM2 or ganglioside GD1a in the specimen (sample to be tested) and comparing the measured value with a reference value set from the measured amount or concentration of ganglioside GM2 or ganglioside GD1a in a healthy subject (sample to be tested). The reference value for healthy subjects or a cutoff value for pancreatic cancer will be determined by controlled clinical trials.

More specifically, when the amount or concentration of GM2 or GD1a in a sample derived from a subject suspected of having pancreatic cancer is significantly higher than the amount or concentration of ganglioside GM2 or ganglioside GD1a in a healthy subject, said subject can be determined to have a high probability of pancreatic cancer.

For example, in the immunohistochemistry or the like, the amount of coloration or luminescence in the tissue is analyzed as an image and they can be compared. In the western blotting or the like, the amount of coloration or luminescence of bands can be analyzed as images and they can be compared. In the enzyme-linked immunosorbent assay, the amount of coloration or luminescence in the liquid is measured and they can be compared.

### [2] Method for Assisting Prediction of Malignancy of Pancreatic Cancer

The method for assisting prediction of malignancy of pancreatic cancer, comprises the steps of: (1) bringing a sample derived from a pancreatic cancer patient into contact with an antibody specific to ganglioside GM2 and/or an antibody specific to ganglioside GD1a, to obtain an antigen-antibody complex, and (2) detecting the antigen-antibody complex.

### (Malignancy)

Pancreatic cancer cells expressing GM2 proliferate in vitro faster than pancreatic cancer cells without GM2 expression. Therefore, pancreatic cancer cells expressing GM2 are expected to increase rapidly in vivo.

In addition, the size of GM2-expressing pancreatic cancer cells increases more rapidly than that of pancreatic cancer cells without GM2 expression, when they are transplanted into nude mice. Therefore, pancreatic cancer cells expressing GM2 are considered to have high malignancy. Furthermore, in the correlation between the stage of pancreatic cancer and GM2, the expression of GM2 is higher in advanced pancreatic cancers.

Similarly, GD1a-expressing cells proliferate faster in nude mice and the expression of GD-1a is higher in advanced-stage pancreatic cancer. That is, pancreatic cancers expressing GD1a have highly malignancy.

### (Sample Derived From Pancreatic Cancer Patient)

In the method for assisting prediction of malignancy of pancreatic cancer of the present invention, the malignancy is predicted by using a sample derived from pancreatic cancer patient. A pancreatic cancer patient can be comprehensively diagnosed by a physician and others, based on symptoms, GM2 or GD1a expression, CT, MRI, and the like. However, samples derived from a subject suspected of having pancreatic cancer are not excluded. Using samples derived from a subject suspected of having pancreatic cancer, the present method may simultaneously assist in the diagnosis of pancreatic cancer and predict the malignancy.

The “sample”, “ganglioside GM2”, “ganglioside GD1a”, “antibody” and the like in the method for assisting prediction of malignancy of pancreatic cancer have the meaning described in the “[1] Method for assisting a diagnosis of pancreatic cancer”. The Contact step (1) and Detection step (2) can also be performed as described in the “[1] Method for assisting a diagnosis of pancreatic cancer.”

If the detected GM2 and/or GD1a in the detection process (2) is above a predetermined value (cutoff value), it can be judged, for example, that “the cancer has highly malignancy” or “the cancer is in progress”. In addition, if it is equal or less to the predetermined value (cutoff value), it can be judged, for example, that “ the cancer has lower malignancy ” or “the cancer has not progressed.” The predetermined value (cut-off value) is determined by the controlled clinical trial.

The cutoff value in the method for assisting prediction of malignancy of pancreatic cancer may be set based on the comparison with the amount or concentration of ganglioside GM2 or ganglioside GD1a in the specimens (samples to be tested) of healthy subjects, or the amount or concentration of ganglioside GM2 or ganglioside GD1a in the specimens (samples to be tested) of relatively benign pancreatic cancers.

### [3] Kit for Diagnosing Pancreatic Cancer or Predicting Malignancy of Pancreatic Cancer

The kit for diagnosing pancreatic cancer or predicting malignancy of pancreatic cancer of the present invention comprises an antibody specific to ganglioside GM2 and/or an antibody specific to ganglioside GD1a, and an agent for detecting an antigen-antibody complex(s) of the antibody and ganglioside GM2 and/or the antibody and ganglioside GD1a.

### (Antibody Specific to Ganglioside GM2)

The antibody specific to ganglioside GM2 included in the kit of the present invention is not particularly limited, as long as it can bind to ganglioside GM2. However, the antibody specific to ganglioside GM2 described in the item of “[1] method for assisting a diagnosis of pancreatic cancer” may be used.

### (Antibody Specific to Ganglioside GD1a)

The antibody specific to ganglioside GD1a included in the kit of the present invention is not particularly limited, as long as it can bind to ganglioside GD1a. However, the antibody specific to ganglioside GD1a described in the item of “[1] method for assisting a diagnosis of pancreatic cancer” may be used.

### (Agent)

The agent included in the kit of the present invention is for detecting the antigen-antibody complex of the antibody and ganglioside GM2 and/or the antibody and ganglioside GD1a. Therefore, the agent is not particularly limited, so long as it is used for detecting the antigen-antibody complex.

For example, when the antibody is not labeled with a labeling material such as an enzyme, the antigen-antibody complex may be detected by a second antibody labeled with an enzyme, biotin, or the like. Therefore, the agent may include the second antibody. In addition, the agent may include an avidin labeled with an enzyme or the like.

Furthermore, the signals of enzymes such as peroxidase, alkaline phosphatase, or β-D-galactosidase which are bound to the antigen-antibody complex can be detected by reacting with a chromogenic or luminescent substrate. Therefore, the agent may comprise a chromogenic substrate or a luminescent substrate. For example, as the substrate for peroxidase, there may be mentioned 3,3′,5,5′-Tetramethylbenzidine (TMB). As the substrate for alkaline phosphatase, there may be mentioned 5-bromo-4-chloro-3-indolyl phosphate (BCIP, X-Phos) and nitro blue tetrazolium chloride (Nitro blue tetrazolium chloride (NBT) which is the oxidizing reagent. As the Substrate for β-D-galactosidase, there may be mentioned 5-bromo-4-chloro-3-indolyl-β-D-galactopyranoside (X-gal) or Fluorescein Di-β-D-galactopyranoside (FDG). Further, as the substrate of luciferase, there may be mentioned luciferin.

Furthermore, the above agents may include a stopping solution to stop the enzyme reaction and a washing solution to wash the solid phase.

### [4] Pharmaceutical Composition

The pharmaceutical composition comprises a GM2 immune complex wherein an anticancer drug or a photosensitizer is bound to an antibody specific to ganglioside GM2, and/or a GD1a immune complex wherein an anticancer drug or a photosensitizer is bound to an antibody specific to ganglioside GD1a.

As shown in FIG. 2, cancer cells can be destroyed by binding immune complexes containing, for example, photosensitizer to GM2-positive or GD1a-positive pancreatic cancer cells in vivo and irradiating them with non-thermal red light.

### (Immune Complex)

The immune complex contained in the pharmaceutical composition of the present invention are complexes consisting of an anticancer drug or a photosensitizer bound to an antibody specific for ganglioside GM2, or an anticancer drug or a photosensitizer bound to an antibody specific for ganglioside GD1a.

### (Antibody)

The antibody which can be used in the pharmaceutical composition of the present invention, is not particularly limited, as long as it is an antibody which specifically bind to GM2 or GD1a, or an antibody fragment having antigen binding site thereof. Preferably, it is a mouse monoclonal antibody, or a chimeric antibody thereof, or a humanized antibody (CDR-grafted antibody) thereof, or a human type antibody.

The chimeric antibody can be obtained as follows. For example, the DNA encoding the heavy chain variable region domain and light chain variable region domain of mouse antibody is linked to the DNA encoding the polypeptide of the constant region of human antibody, and it is then inserted into an expression vector, and it is introduced into the host for production. The heavy chain variable region domain and light chain variable region domain used in the chimeric antibody are not limited. The origin of the polypeptides of the constant region is not limited, as long as it is human antibody. For example, the chimeric antibody can be obtained by the heavy chain variable region domain and light chain variable region domain of mouse IgG, and the polypeptide of the constant region of human IgM or IgG.

In the humanized antibodies (CDR-grafted antibodies), the complementarity-determining regions (CDR) of, for example, human antibodies are replaced with the complementarity-determining regions (CDR) of, for example, mouse antibodies, and the complementarity-determining regions (CDR) of mouse antibodies are transplanted. Specifically, a DNA sequence designed to link the CDRs of a mouse antibody to the framework regions (FRs; framework regions) of a human antibody is synthesized by PCR from several oligonucleotides with overlapping portions at the ends. The resulting DNA is concatenated with DNA encoding the C region of the human antibody, incorporated into an expression vector, and then introduced into a host for production, to obtain the humanized antibody. An origin of polypeptides of the complementarity-determining region, the framework region, and the constant region used for humanized antibodies is not particularly limited, as long as it is human antibody. For example, the humanized antibody can be obtained from the complementarity-determining region of mouse IgG, and the framework region and the constant region of human IgM or IgG. Further, the antigen-binding fragments of humanized antibodies can be obtained from the complementarity-determining region of mouse and the human IgM or IgG framework region of human.

The human type antibody can be obtained from the transgenic animals transfected with human antibody genes, or it is a monoclonal antibody obtained by cell fusion of human antibody-producing cells with myeloma cells. As the method for obtaining the human type antibody, a technique of obtaining human antibodies by panning using a human antibody library is known, in addition to the above methods of obtaining from transgenic animals or by cell fusion of human antibody-producing cells. For example, the variable regions of human antibodies can be expressed as single chain antibodies (scFv) on the surface of phages by phage display method, and phages that bind to the antigen can be selected. By analyzing the genes of the selected phages, it is possible to determine the DNA sequence encoding the variable regions of the human antibodies that bind to the antigen. By determining the DNA sequence of the scFv that binds to the antigen, an appropriate expression vector can be created and human antibodies can be obtained.

### (Anticancer Drug)

The anticancer drug used in the immune complex is not particularly limited, as long as it is effective for pancreatic cancer, but there may be mentioned tegafur, gimeracil, oteracil potassium, gemcitabine (Gemzar), fluorouracil [5-FU], calcium levofolinate, irinotecan, oxaliplatin, nab-paclitaxel (Abraxane), or erlotinib (Tarceva). It is also possible to bind radioisotopes to antibodies and use them as immune complexes.

### (Photosensitizer)

As the photosensitizer, there may be mentioned porphyrin compounds (e.g., 5-aminolevulinic acid), proline compounds (e.g., proline), bacterioproline compounds, or phthalocyanine compounds (e.g., phthalocyanine). Further, photophrin, laserphyrin, aminolevulinic acid (ALA), silicon phthalocyanine Pc4, m-tetrahydroxyphenylchlorin (mTHPC), chlorin e6 (Ce6), almera, leblanc, foscan, metvix, hexvix, photochlor, photosens, photorex, lumacan, bisonac, amfinex, Verteporfin, purlytin, ATMPn, zinc phthalocyanine (ZnPc), protoporphyrin IX (PpIX), pyropheophorbide a (PPa), pheophorbide (PhA), or the like can also be used.

For example, phthalocyanine (IR700) is chemically changed by near-infrared light (non-thermal red light) at 700 nm. That is to say, the cancer cells are damaged (expansion, destruction, or necrosis) by absorbing light energy and generating heat. Near-infrared light can also be irradiated by guiding an optical fiber near the pancreatic cancer. Furthermore, it can be designed so that the immune complex (antibody-phthalocyanine conjugate) is activated by near-infrared irradiation only when it binds to the target molecule (GM2 or GD1a).

Cancer-specific proteins released from cancer cells destroyed by the pharmaceutical composition of the present invention sensitize and proliferate cytotoxic T cells against cancer as antigens, then, the number of cytotoxic T cells attacking cancer is increased thereby. The pharmaceutical compositions of the present invention can efficiently treat pancreatic cancer by such mechanism as well.

The formulation of the pharmaceutical composition of the present invention is not particularly limited. For example, there may be mentioned, oral agents, such as powders, subtle granules, granules, tablets, capsules, suspensions, emulsions, sylups, extracts, or balls; or injections or the like, but the injections are preferable. For example, in a preparation of the injections, an aqueous solvent such as normal saline solution or Ringer solution, non-aqueous solutions such as plant oil or fatty acid ester, a tonicity agent such as glucose or sodium chloride, a solubility assisting agent, a stabilizing agent, an antiseptic agent, a suspending agent, or an emulsifying agent, may be optionally used, in addition to the active ingredient.

The pharmaceutical composition of the present invention may contain, but is not limited to, 0.01 to 99% by weight, preferably 0.1 to 80% by weight, of the active ingredient. A dose (therapeutic effective amount) of the pharmaceutical composition of the present invention may be appropriately determined in accordance with, for example, the type of each active ingredient (anticancer drug or photosensitizer), the type or stage of pancreatic cancer, age, sex, body weight, or degree of symptom of each patient, route of administration, or the like, and the determined dosage can be administered orally or parenterally.

The pharmaceutical composition of the present invention can be used in a method of treating pancreatic cancer, which is characterized by administering a therapeutically effective amount of the pharmaceutical composition to a pancreatic cancer patient.

### [5] Method for Determining Therapeutic Effect

In the method for determining therapeutic effect, the therapeutic efficacy of the pharmaceutical composition for treating pancreatic cancer is determined. The therapeutic effect can be determined by measuring, for example, an anticancer drug or degradation products thereof, or a photosensitizer or degradation products thereof.

For example, when a conjugate of the antibody and the photosensitizer (immune complex) is bound to GM2 or GD1a, the therapeutic effect of the pharmaceutical composition for treating pancreatic cancer can be measured by measuring the released photosensitizer or degradation product thereof, in the immune complex designed to be activated by near-infrared light.

### EXAMPLES

The present invention now will be further illustrated by, but is by no means limited to, the following Examples.

### Example 1

In this Example, the expression of ganglioside GM2 in cultured cells derived from eight types of pancreatic cancer is examined.

Fluorescent antibody staining was performed using mouse anti-GM2 antibody (TCI). The cells were reacted with the GM2 antibody, and then fluorescently stained using a fluorescently labeled secondary antibody (Molecular Probes). The percentage of cells expressing ganglioside GM2 was calculated using FACSAria Cell Sorter (Becton Dickinson). As shown in Table 1 and FIG. 3(A), 21.4% of MIA PaCa-2 cells were positive for GM2.

Furthermore, the fluorescent-stained cells were sorted by a cell sorter, and GM2-positive cells and GM2-negative cells can be separated as shown in FIG. 3(B).

TABLE 1

| Cell | Rate of GM2 positive cells |
|---|---|
| PANC-1 | 2% |
| T3M-4 | 0% |
| PK-59 | 2.8% |
| PK-45P | 0% |
| MIA PaCa-2 | 21.4% |
| PK-8 | 0% |
| PK-1 | 7% |
| KP-4 | 4.7% |

### Example 2

In this Example, the expression of ganglioside GD1a in cultured cells derived from eight types of pancreatic cancer is examined.

Fluorescent antibody staining was performed using mouse anti-GD1a antibody (TCI). The cells were reacted with the GD1a antibody, and then fluorescently stained using a fluorescently labeled secondary antibody (Molecular Probes). The percentage of cells expressing ganglioside GD1a was calculated using FACSAria Cell Sorter (Becton Dickinson). As shown in Table 4, the expression of GD1a was high in seven types of cells other than MIA PaCa-2 cells, especially in PK-59 cells, PK-45P cells, and PK-1 cells.

### Example 3

In this example, the growth rate of GM2-positive cells was examined.

GM2-positive MIA PaCa-2 cells and GM2-negative MIA PaCa-2 cells, obtained in Example 1, were individually seeded into 96-well plates at a concentration of 5×10³ cells/well in RPMI-1640 medium and cultured for 72 hours. Adherent cells were incubated with WST-8 cell counting reagent (Wako) for 2 hours and absorbance was measured at 450 nm. As shown in FIG. 5, the proliferation rate of GM2-positive cells was higher than that of GM2-negative cells. Therefore, GM2-positive cells were considered to have a high malignancy.

### Example 4

In this example, the sensitivity of GM2-expressing cells to anticancer drugs (gemcitabine, fluorouracil [5-FU], and Abraxane) were examined.

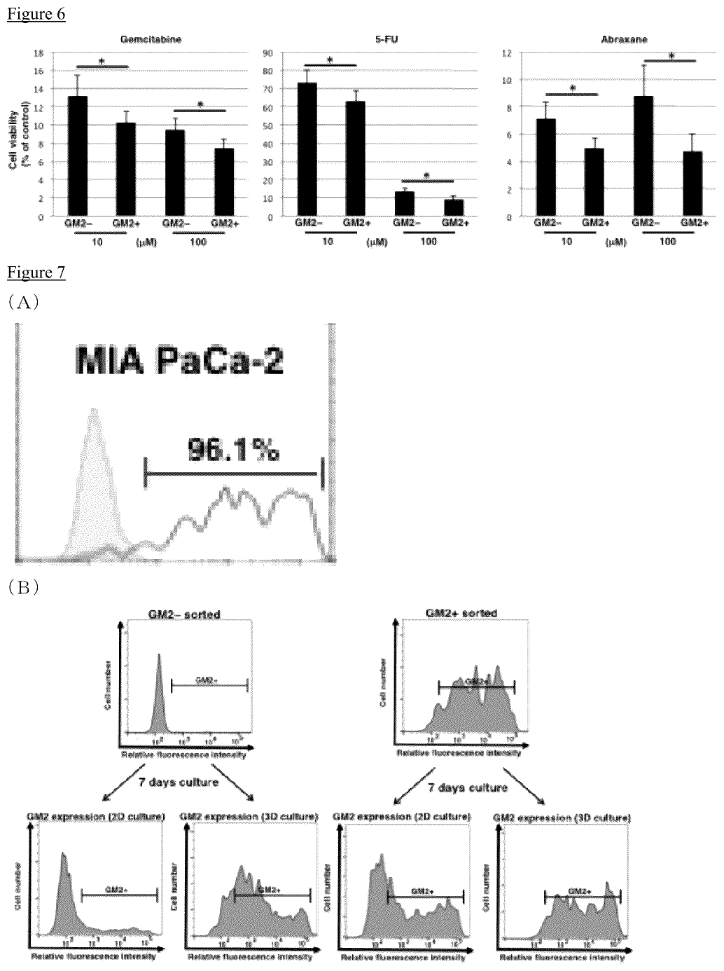

MIA PaCa-2 cells (3.0×10³ cells/well) were seeded in 96-well plates. After 1 day, each anticancer drug was added thereto at a concentration of 10 μM or 100 μM. The cells were incubated for 3 days, and the proliferation rate of the cells was measured by ATP assay. In all cases of gemcitabine, fluorouracil [5-FU], and Abraxane, the proliferation rate of GM2-positive cells was lower than that of GM2-positive cells, suggesting that the anticancer drugs were effective against GM2-positive cells (FIG. 6).

### Example 5

In this example, the changes of GM2 expression in three-dimensional culture was examined.

MIA PaCa-2 cells were cultured on ultra-low attachment plates at a concentration of 1.0×10⁴ cells/well (24-well plate: Corning) for 7 days. Cell clusters were obtained. The expression of GM2 on the cell surface was examined, and 96.1% of the cells were GM2-positive cells, as shown in FIG. 7(A). Therefore, the number of GM2-positive cells increased in three-dimensional culture.

Furthermore, MIA PaCa-2 cells were separated into GM2-positive cells and GM2-negative cells by the same method as in Example 1, and three-dimensionally cultured in the same manner. As shown in FIG. 7(B), GM2-positive cells increased more than GM2-negative cells in the three-dimensional culture. In addition, by two-dimensional culture of GM2-positive cells, GM2-negative cells and GM2-positive cells proliferated.

### Example 6

In this example, the effects of AMP-dNM or MAP kinase inhibitor on GM2 expression were examined.

Under the same conditions as in Example 5, MIA PaCa-2 cells were three-dimensionally cultured, and the expression of GM2 was measured by adding AMP-dNM (10 μM) which is an inhibitor of glycolipid synthesis, or PD0325901 (1 μM) which is a MAP kinase inhibitor.

As shown in FIG. 8(A), GM2 expression was decreased by the addition of AMP-dNM. As shown in FIG. 8(B), microvilli were hardly observed in the cell clusters, and vacuoles indicated by stars were conspicuously observed by the addition of AMP-dNM.

As shown in FIG. 8(C), the expression of GM2 was decreased by the addition of PD0325901. In addition, as shown in FIG. 8(D), the size of cell clusters was clearly reduced.

### Example 7

In this example, the growth of GM2-expressing cells in nude mice was examined.

Female 9-week-old BALB/c nude mice (5 mice each) were inoculated with 1×10⁵ GM2-positive or GM2-negative cells per mouse. Tumor size was calculated every week for 5 weeks according to the following formula.

$$Size ( volume ) = a \times b 2 \times 0.5$$

(in the formula, a is the longest diameter and b is the shortest diameter)

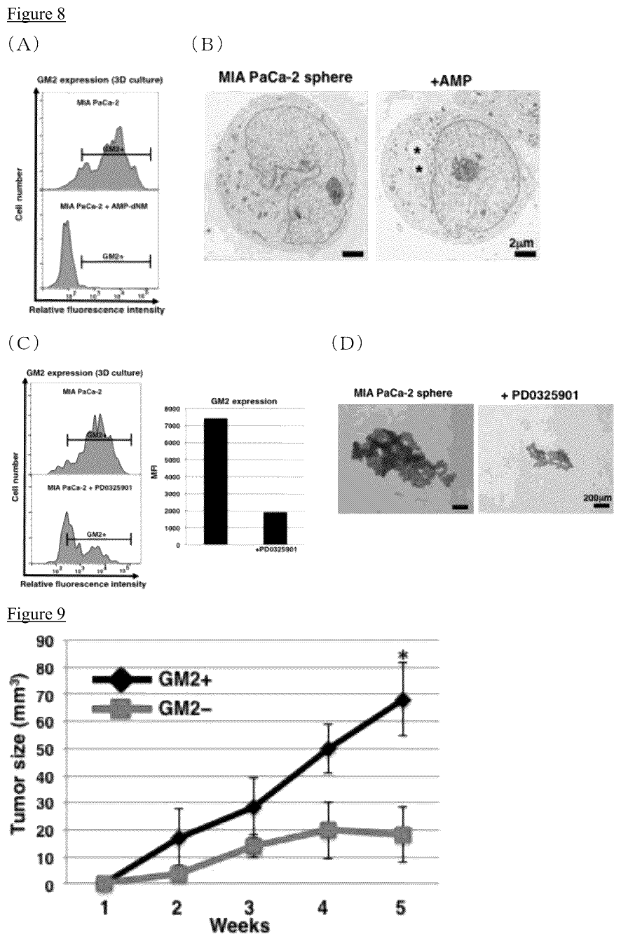

As shown in FIG. 9, the tumor mass of GM2-positive cells was about 3.5 times larger than that of GM2-negative cells in 5 weeks.

### Example 8

In this example, the expression of GM2 in human pancreatic cancer tissues was examined. A block of pathological tissue was cut into 4-μm-thick sections and stained with anti-GM2 antibody (1:1000 dilution; TCI).

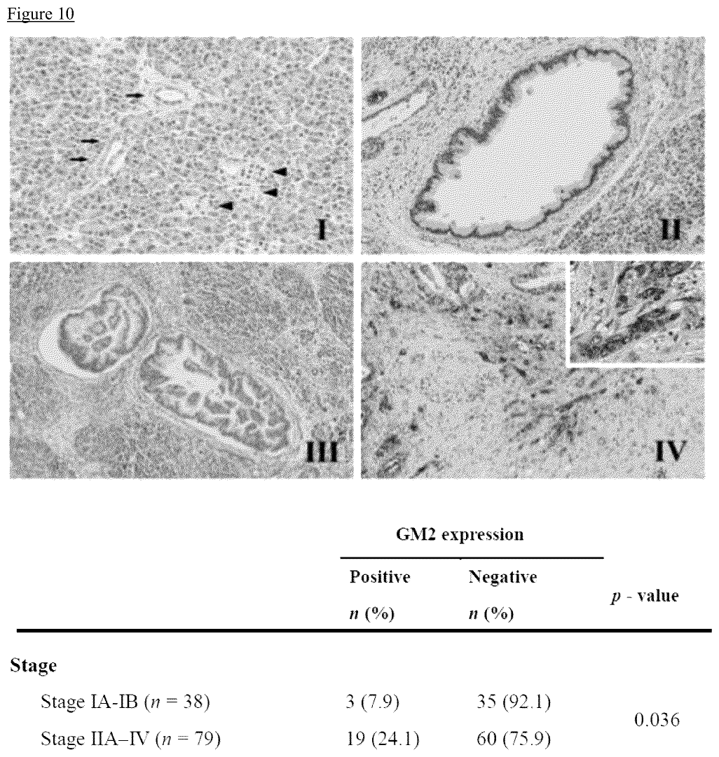

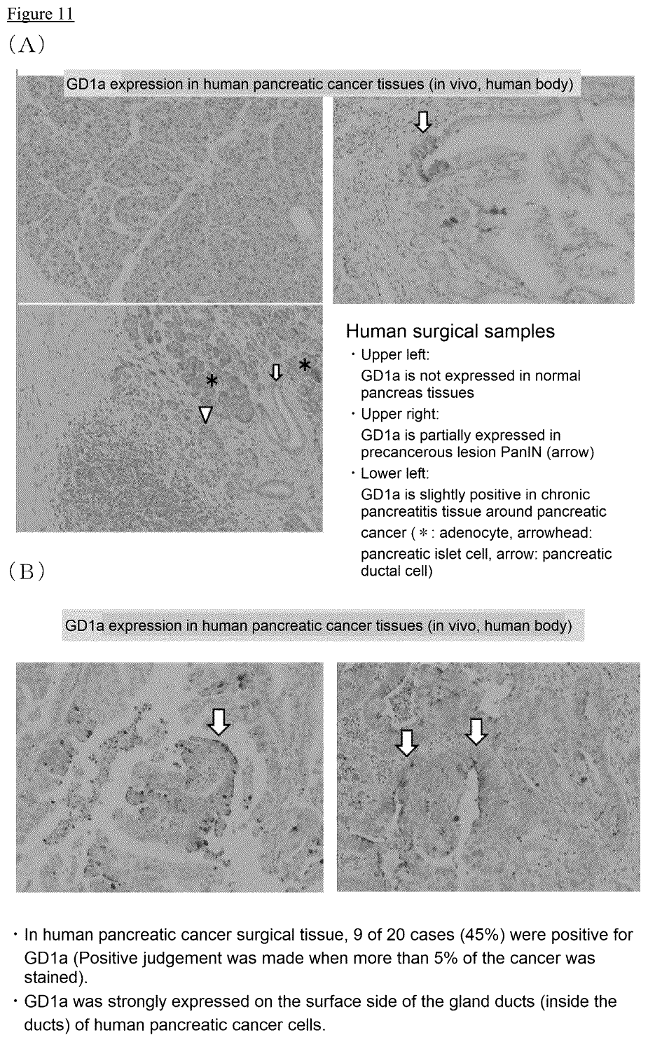

As shown in FIG. 10, the positivity of GM2 was 7.9% in 38 specimens of stage IA-IB. While, the positivity of GM2 was 24.1% in 79 specimens of stage IIA-IV, indicating that the positivity of GM2 increased as the stage progressed. GM2 expression was determined to be positive when 5% of the cancer cells in the tissue were stained.

### Example 9

### Expression of GD1a in Pancreatic Cancer Tissues

In this example, the expression of GD1a in human pancreatic cancer tissues was examined. A block of pathological tissue was cut into 4-μm-thick sections and stained with anti-GD1a antibody (1:1000 dilution; TCI).

As shown in FIG. 11, no GD1a expression was observed in human normal pancreatic tissues, while 45% (9/20 cases) of surgical tissues of pancreatic cancer were positive for GD1a.

### Example 10

In this example, the induction of cell death by anti-GM2 or anti-GD1a antibody targeting GM2-or GD1a-expressing cells and by a second immunotoxin in which a secondary antibody labeled with saporin, was examined.

MIA PaCa-2 cells (2.0×10³ cells/well) expressing GM2 were seeded in 96-well plates. After 1 day, anti-GM2 antibody of a concentration of 5 ng/ml and secondary antibody (anti-M-ZAP) of a concentration of 0.2 ng/ml were added. The cells were cultured for 3 days, and the proliferation rate of the cells was measured by ATP assay.

PK-8 cells expressing GD1a were reacted with anti-GD1a antibody and then with secondary antibody (anti-M-ZAP) and seeded at 2.0×10³ cells/well in 96-well plates. Cells were incubated for 3 days, and the proliferation rate was measured by ATP assay.

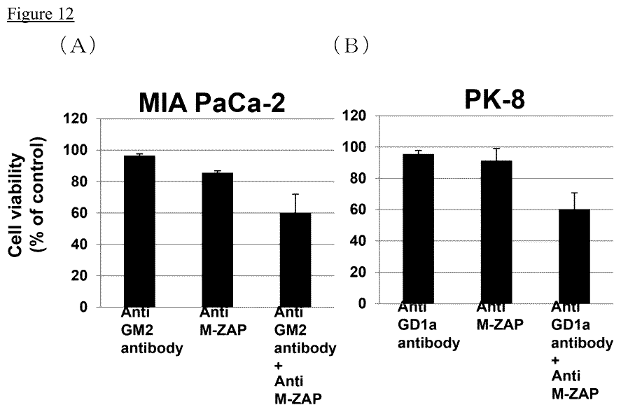

Both MIA PaCa-2 cells and PK-8 cells showed poor proliferation rates in the cells reacted with primary and secondary antibodies, suggesting that each antibody can induce cell death by targeting GM2 or GD1a, respectively (FIG. 12).

### INDUSTRIAL APPLICABILITY

The method for assisting a diagnosis of pancreatic cancer of the present invention can be used for assisting early diagnosis of pancreatic cancer. The method for assisting prediction of malignancy of pancreatic cancer of the present invention can predict malignancy of pancreatic cancer. The pharmaceutical composition of the present invention can be used for treating pancreatic cancer.

1. A method for assisting a diagnosis of pancreatic cancer, comprising the steps of:
(1) bringing a sample derived from a subject suspected of having pancreatic cancer into contact with an antibody specific to ganglioside GD1a, to obtain an antigen-antibody complex, and
(2) detecting the antigen-antibody complex.

2. A method for assisting prediction of malignancy of pancreatic cancer, comprising the steps of:
(1) bringing a sample derived from a pancreatic cancer patient into contact with an antibody specific to ganglioside GD1a, to obtain an antigen-antibody complex, and
(2) detecting the antigen-antibody complex.

3. A kit for diagnosing pancreatic cancer, or predicting malignancy of pancreatic cancer, comprising an antibody specific to ganglioside GD1a, and an agent for detecting an antigen-antibody complex(s) of the antibody and ganglioside GD1a.

4. A pharmaceutical composition for treating pancreatic cancer, comprising a GD1a immune complex wherein an anticancer drug or a photosensitizer is bound to an antibody specific to ganglioside GD1a.

5. The pharmaceutical composition for treating pancreatic cancer according to claim 4, wherein the photosensitizer is phthalocyanine.

6. The pharmaceutical composition for treating pancreatic cancer according to claim 4, wherein the antibody is a humanized antibody.

7. A method for determining a therapeutic effect of the pharmaceutical composition for treating pancreatic cancer according to claim 4.
